(19) United States
(12) Patent Application Publication
(10) Pub. No.: US 2025/0006294 A1
(43) Pub. Date: Jan. 2, 2025

(54) CHARGING PROCESSING DEVICE, CHARGING PROCESSING METHOD, AND RECORDING MEDIUM

(71) Applicant: NEC Corporation, Tokyo (JP)

(72) Inventors: Takeharu KITAGAWA, Tokyo (JP); Yusuke Kobayashi, Tokyo (JP)

(73) Assignee: NEC Corporation, Tokyo (JP)

(21) Appl. No.: 18/738,305

(22) Filed: Jun. 10, 2024

(30) Foreign Application Priority Data
Jun. 28, 2023 (JP) 2023-105624

(51) Int. Cl. G16B 15/20; G16B 20/00

(52) U.S. Cl. CPC G16B 15/20; G16B 20/00

(57) ABSTRACT

A charging processing device according to the present disclosure includes an acquisition means for acquiring primary sequence data of amino acids constituting a protein, a prediction means for predicting a three-dimensional structure of the protein based on the primary sequence data, an output means for outputting a moving image in a mode in which the three-dimensional structure is graspable, a purchase reception means for receiving a purchase of prediction result data for the three-dimensional structure from a customer, and a charging means for executing charging processing with respect to the customer when the purchase is received.

This application is based upon and claims the benefit of priority from Japanese Patent Application No. 2023-105624, filed on Jun. 28, 2023, the disclosure of which is incorporated herein in its entirety by reference.

### TECHNICAL FIELD

The present disclosure relates to a charging processing device, a charging processing method, a program, and the like.

### BACKGROUND ART

There is a technology for predicting a three-dimensional structure of a protein from a sequence structure of amino acids using a machine learning model.

For example, JP 2022-501696 A discloses that a final predicted structure of a given protein is determined by a data processing device implementing a machine learning model.

### SUMMARY

An example of an object of the present disclosure is to provide a charging processing device capable of charging a customer when the customer considers a prediction result to be worth purchasing.

A charging processing device according to an aspect of the present disclosure includes an acquisition means for acquiring primary sequence data of amino acids constituting a protein, a prediction means for predicting a three-dimensional structure of the protein based on the primary sequence data, an output means for outputting a moving image in a mode in which the three-dimensional structure is graspable, a purchase reception means for receiving a purchase of prediction result data for the three-dimensional structure from a customer, and a charging means for executing charging processing with respect to the customer when the purchase is received.

A charging processing method according to an aspect of the present disclosure, performed by a computer, includes acquiring primary sequence data of amino acids constituting a protein, predicting a three-dimensional structure of the protein based on the primary sequence data, outputting a moving image in a mode in which the three-dimensional structure is graspable, receiving a purchase of prediction result data for the three-dimensional structure from a customer, and executing charging processing with respect to the customer when the purchase is received.

A program according to an aspect of the present disclosure causes a computer to execute acquiring primary sequence data of amino acids constituting a protein, predicting a three-dimensional structure of the protein based on the primary sequence data, outputting a moving image in a mode in which the three-dimensional structure is graspable, receiving a purchase of prediction result data for the three-dimensional structure from a customer, and executing charging processing with respect to the customer when the purchase is received.

### BRIEF DESCRIPTION OF THE DRAWINGS

Exemplary features and advantages of the present invention will become apparent from the following detailed description when taken with the accompanying drawings in which:

FIG. 1 is a diagram illustrating a configuration including a charging processing device according to the present disclosure;

FIG. 2 is a diagram illustrating a hardware configuration in which the charging processing device according to the present disclosure is achieved by a computer device and its peripheral device;

FIG. 3A is a diagram illustrating an example in which an output unit outputs a moving image of a three-dimensional structure;

FIG. 3B is a diagram illustrating an example in which the output unit outputs a moving image of a three-dimensional structure;

FIG. 3C is a diagram illustrating an example in which the output unit outputs a moving image of a three-dimensional structure;

FIG. 3D is a diagram illustrating an example in which the output unit outputs a moving image of a three-dimensional structure;

FIG. 4A is a diagram illustrating an example in which the output unit outputs a moving image of a three-dimensional structure;

FIG. 4B is a diagram illustrating an example in which the output unit outputs a moving image of a three-dimensional structure;

FIG. 4C is a diagram illustrating an example in which the output unit outputs a moving image of a three-dimensional structure;

FIG. 4D is a diagram illustrating an example in which the output unit outputs a moving image of a three-dimensional structure;

FIG. 5 is a flowchart illustrating a charging processing operation according to the present disclosure;

FIG. 6 is a diagram illustrating a configuration including a charging processing device according to the present disclosure; and

FIG. 7 is a flowchart illustrating a charging processing operation according to the present disclosure.

### EXAMPLE EMBODIMENT

Next, example embodiments will be described in detail with reference to the drawings.

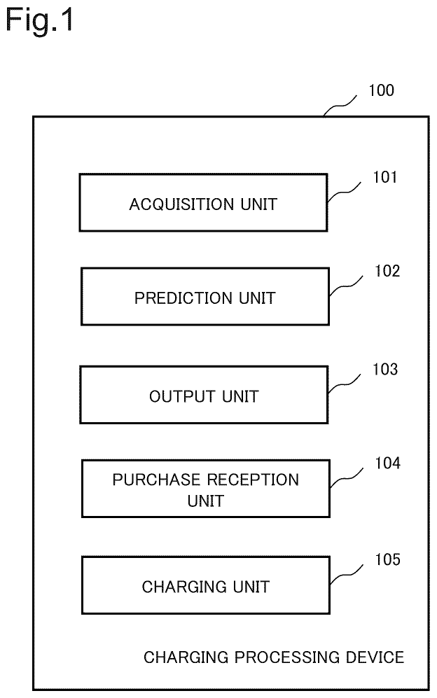

FIG. 1 is a diagram including a configuration of a charging processing device 100 according to the present disclosure. Referring to FIG. 1, the charging processing device 100 includes an acquisition unit 101, a prediction unit 102, an output unit 103, a purchase reception unit 104, and a charging unit 105. The charging processing device 100 is a device for predicting a three-dimensional structure of a protein from a primary sequence of amino acids and performing charging processing when a customer purchases prediction result data.

For example, when primary sequence data of amino acids of a protein to be predicted is input, the charging processing device 100 outputs a moving image in a mode in which a three-dimensional structure of the protein is graspable. The customer determines whether to purchase prediction result data for the three-dimensional structure using the moving image as a determination material. Then, the charging processing device 100 executes charging processing when the customer purchases the prediction result data.

In the present example embodiment, examples of proteins to be predicted include human or animal/plant organs (hearts and brains), human or animal/plant tissues (retinas and blood vessels), pollen (about 30 μm in size), cells (6 to 25 μm), cell nuclei (6 μm), bacteria (1 to 5 μm), chromosomes (1 to 2 μm), high molecular proteins (several tens to several hundreds of nm), viruses (100 nm), antibodies (10 to 15 nm), low molecular proteins (several to several tens of nm), enzymes (3 to 5 nm), and the like. These proteins are examples, and the proteins to be predicted are not limited thereto. The protein to be predicted may be a protein alone or a protein complex. The prediction target may be a protein in which an antigen such as a specific bacterium or virus and an antibody such as a drug are bound to each other. In this case, a docking state between the antigen and the antibody can be checked. Furthermore, the protein to be predicted may be a protein in which a coronavirus and a drug are bound to each other. In particular, the prediction target may be docking of a drug to a spike protein on a coronavirus surface or a structural change of the spike protein due to the docking. Other examples of proteins to be predicted include binding between a protein and a ligand complex, and binding between an enzyme contained in a plant and a pesticide.

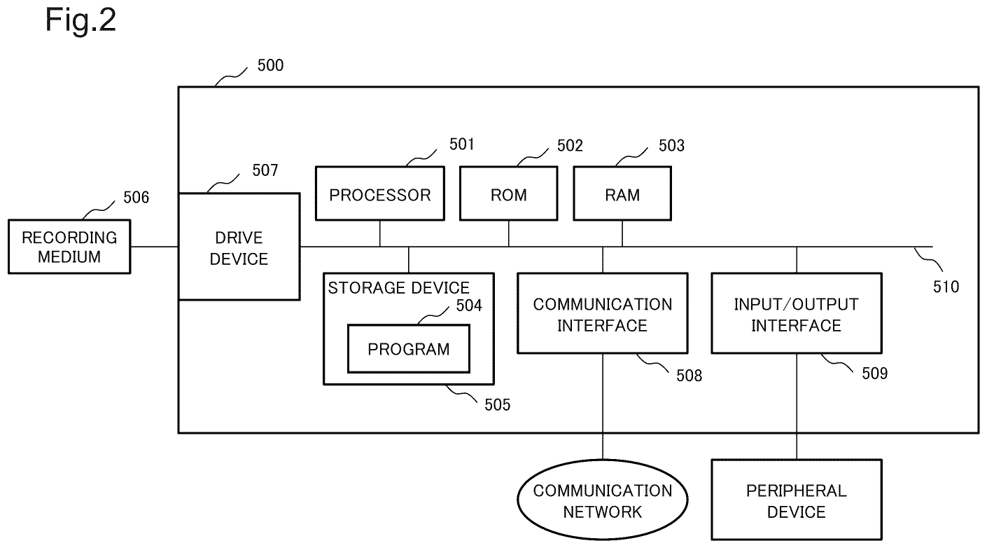

FIG. 2 is a diagram illustrating an example of a hardware configuration in which the charging processing device 100 according to the present disclosure is achieved by a computer device 500 including a processor. As illustrated in FIG. 2, the charging processing device 100 includes a processor 501, memories such a read only memory (ROM) 502 and a random access memory (RAM) 503, a storage device 505 such as a hard disk that stores a program 504, a communication interface 508 for network connection, and an input/output interface 509 that inputs and outputs data. As the processor 501, a central processing unit (CPU), a graphics processing unit (GPU), a combination thereof, or the like can be used.

In a first example embodiment, the components in the charging processing device 100 are connected to each other, for example, via a bus 510. The charging processing device 100 according to the first example embodiment illustrated in FIG. 1 may be configured by cloud computing or the like.

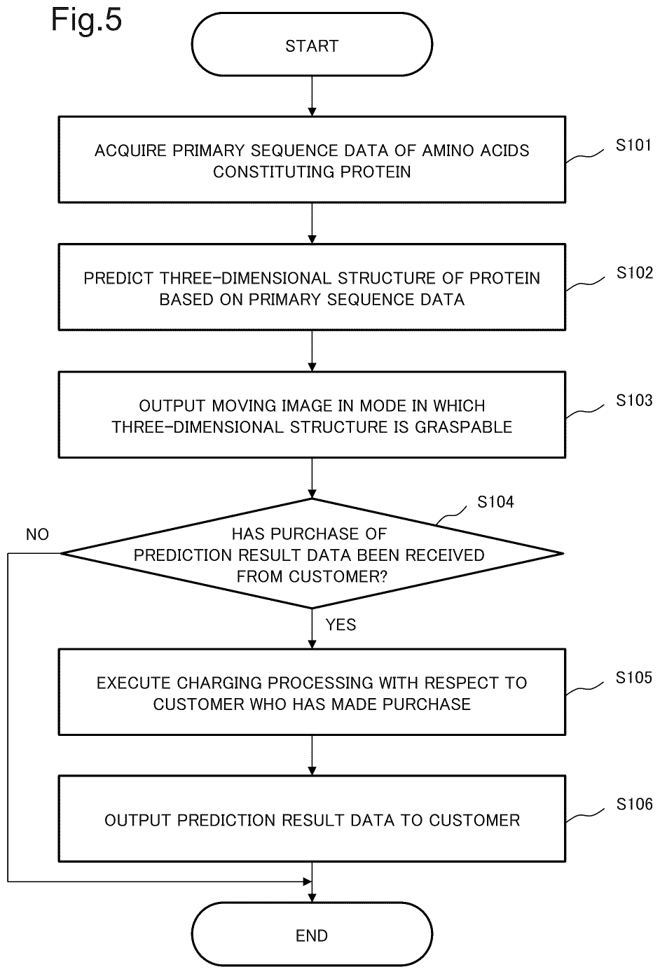

The processor 501 operates an operating system to control the entire charging processing device 100. In addition, the processor 501 reads a program or data from a recording medium 506 mounted on, for example, a drive device 507 to a memory. In addition, the processor 501 functions as the acquisition unit 101, the prediction unit 102, the output unit 103, the purchase reception unit 104, and the charging unit 105, or some of them, and executes a process or a command of a flowchart illustrated in FIG. 5 to be described below based on the program.

The recording medium 506 is, for example, an optical disk, a flexible disk, a magneto-optical disk, an external hard disk, a semiconductor memory, or the like. The semiconductor memory or the like, which is a part of the recording medium, is a non-volatile storage device, and records the program therein. A program may be downloaded from an external computer (not illustrated) connected to a communication network.

As described above, the first example embodiment illustrated in FIG. 1 is implemented by the computer hardware illustrated in FIG. 2. However, the means for implementing each of the units included in the charging processing device 100 of FIG. 1 is not limited to the configuration described above. In addition, the charging processing device 100 may be implemented by one physically coupled device, or may be implemented by two or more physically separated devices by connecting the plurality of devices to each other in a wired or wireless manner. The charging processing device 100 may further include a display device such as a display that displays a moving image having a three-dimensional structure. In addition, the charging processing device 100 and the display device may be configured as a system implemented by separate devices.

The acquisition unit 101 is a means for acquiring primary sequence data of amino acids constituting a protein. The primary sequence data is data in FASTA format or the like indicating an order in which amino acids constituting a protein are arranged. The format is not limited thereto as long as the primary sequence of the protein is graspable from the data. The acquisition unit 101 acquires, for example, primary sequence data stored by a customer in a server prepared for predicting a three-dimensional structure. When acquiring the primary sequence data, the acquisition unit 101 outputs the primary sequence data to the prediction unit 102.

The prediction unit 102 is a means for predicting a three-dimensional structure of the protein based on the primary sequence data. The prediction unit 102 predicts the three-dimensional structure, for example, using a learning model obtained by learning how amino acids of proteins whose shapes have already been known are connected to each other, using three-dimensional structures disclosed in a database of known proteins as teacher data. This learning model may be constituted by a neural network having a complicated multilayer structure. The prediction unit 102 may constituted by a publicly available structure prediction tool. For example, the prediction unit 102 inputs primary sequence data to the structure prediction tool, calculates a distance between amino acids constituting a protein, a bent angle of the protein, etc., and outputs a predicted three-dimensional structure. The number of pieces of data on three-dimensional structure output by the prediction unit 102 may be plural (for example, 2 to 5 types), and the number of pieces of output data can be changed on the structure prediction tool. In this case, the prediction unit 102 may output a three-dimensional structure predicted using different learning models with a vary reliability of the three-dimensional structure to be output.

The output unit 103 is a means for outputting a moving image in a mode in which the three-dimensional structure is graspable. The mode in which the three-dimensional structure is graspable is, for example, a mode in which the three-dimensional structure such as a helical structure, a folded structure, or a spherically rounded structure of the protein can be visually recognized.

In a case where the moving image is a planar (2D) image, images allowing the three-dimensional structure to be visually recognized from a plurality of directions are necessary, and for example, images obtained by photographing the three-dimensional structure from six directions including front, rear, left, right, and top and bottom views and an image obtained by photographing the three-dimensional structure from an angle of 45 degrees are necessary. In a case where the moving image is a video image, for example, the video image is obtained by rotating the three-dimensional structure about each of the axes orthogonal to each other. Specifically, the output unit 103 may output a video image obtained by rotating the three-dimensional structure about each of the horizontal axis and the vertical axis on a screen displaying the three-dimensional structure. Furthermore, the output unit 103 may output a plurality of images cut out from these video images when the three-dimensional structure is oriented in a predetermined direction. The output unit 103 displays the moving image on a display device such as a display connected to the charging processing device 100. In addition, the output unit 103 may transmit a moving image file to a terminal possessed by the customer. An example of a format of the moving image file is MP4.

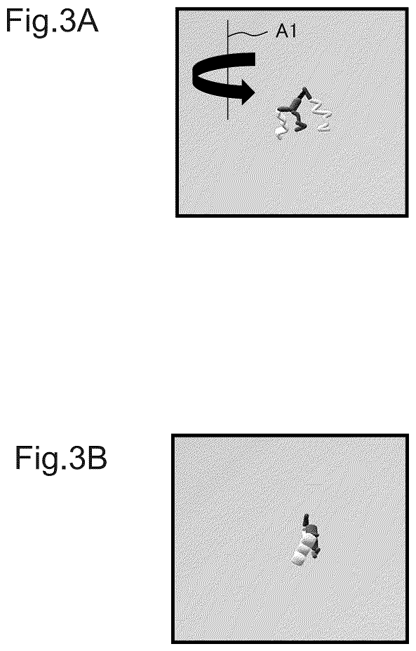

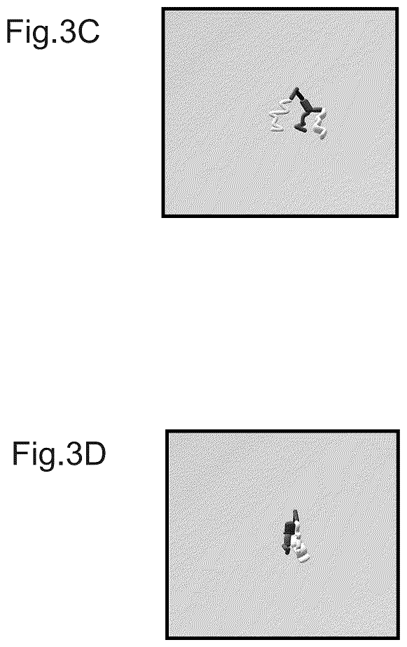

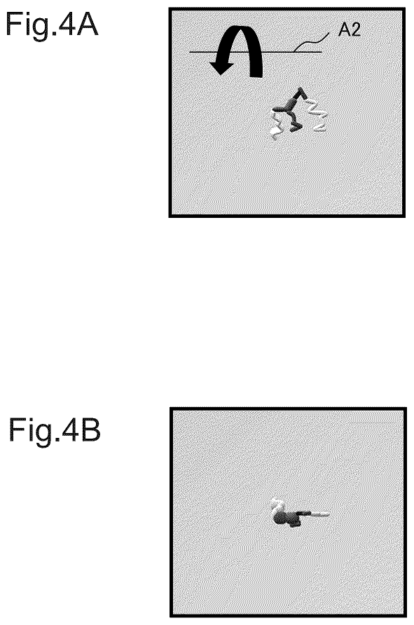

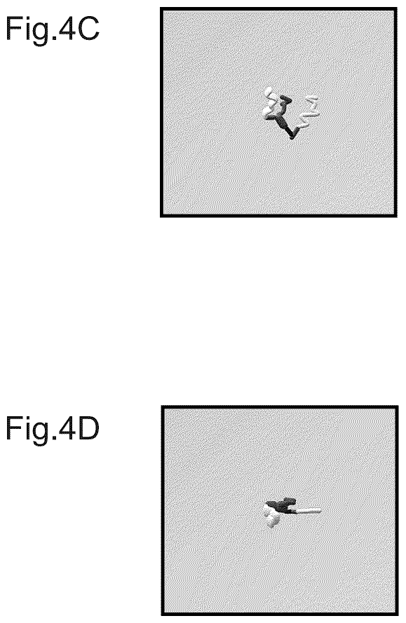

FIGS. 3A to 3D and FIGS. 4A to 4D are diagrams each illustrating an example in which a moving image of a three-dimensional structure of a protein is output by the output unit 103. FIG. 3A is an image when the three-dimensional structure is rotated counterclockwise about the vertical axis (A1). FIG. 3B is an image cut out when the left surface of the three-dimensional structure in FIG. 3A becomes oriented in the front direction, FIG. 3C is an image cut out when the rear surface of the three-dimensional structure in FIG. 3A becomes oriented in the front direction, and FIG. 3D is an image cut out when the right surface of the three-dimensional structure in FIG. 3A becomes oriented in the front direction. FIG. 4A is an image when the three-dimensional structure is rotated around the horizontal axis (A2). FIG. 4B is an image cut out when the top surface of the three-dimensional structure in FIG. 4A becomes oriented in the front direction, FIG. 4C is an image cut out when the rear surface of the three-dimensional structure in FIG. 4A becomes oriented in the front direction, and FIG. 4D is an image cut out when the bottom surface of the three-dimensional structure in FIG. 4A becomes oriented in the front direction. The images illustrated in FIGS. 3A to 3D and FIGS. 4A to 4D are examples of moving images output by the output unit 103, and the moving images output by the output unit 103 are not limited thereto.

The purchase reception unit 104 is a means for receiving a purchase of prediction result data for the three-dimensional structure from the customer. The purchase reception unit 104 receives a request for purchasing prediction result data from the customer. The form of the request for purchase received by the purchase reception unit 104 is not particularly limited, and a selection of a purchase made on a screen where the moving image is displayed by the output unit 103 may be received, or information including a request for purchase may be received from the terminal possessed by the customer. In addition, when receiving the request for purchase, the purchase reception unit 104 may receive information for identifying the customer, payment information necessary for charging the customer, and the like.

The charging unit 105 is a means for executing charging processing with respect to the customer who has purchased prediction result data. The charging unit 105 performs charging processing based on the received payment information. Specifically, the charging unit 105 proceeds with payment processing for the purchase cost of the prediction result data for the three-dimensional structure to be purchased by a known method using the payment information of the customer registered in advance.

In the present example embodiment, the output unit 103 may further output the prediction result data for the three-dimensional structure to the customer when the purchase is received. The prediction result data is file forming data capable of displaying a three-dimensional structure or outputting a moving image file for a three-dimensional structure on a terminal possessed by a customer, and is, for example, data in a protein data bank (PDB) format. The timing at which the output unit 103 outputs the prediction result data for the three-dimensional structure to the customer is not particularly limited as long as the prediction result data is output after the purchase reception unit 104 receives the purchase of the prediction result data. For example, the output unit 103 may output the prediction result data for the three-dimensional structure after the charging processing is completed by the charging unit 105. However, in the charging processing device 100, the configuration in which the output unit 103 outputs the prediction result data to the customer is not included in the minimum configuration necessary for solving the problem of the invention according to the present disclosure.

An operation of the charging processing device 100 configured as described above will be described with reference to a flowchart of FIG. 5.

FIG. 5 is a flowchart illustrating an outline of an operation of the charging processing device 100 according to the present disclosure. Note that the process according to this flowchart may be executed based on the program control by the processor described above.

As illustrated in FIG. 5, first, the acquisition unit 101 acquires primary sequence data of amino acids constituting a protein (step S101). Next, the prediction unit 102 predicts a three-dimensional structure of the protein based on the primary sequence data (step S102). Next, the output unit 103 outputs a moving image in a mode in which the three-dimensional structure is graspable (step S103). Next, when the purchase reception unit 104 has received a purchase of prediction result data for the three-dimensional structure from a customer (S104; YES), the charging unit 105 executes charging processing with respect to the customer who has purchased the prediction result data (step S105), and the output unit 103 outputs the prediction result data to the customer (step S106). On the other hand, when the purchase reception unit 104 has not received a purchase of prediction result data for the three-dimensional structure from the customer (S104; NO), the flow ends. In this manner, the charging processing device 100 ends the charging processing operation.

In the charging processing device 100 described above, the output unit 103 outputs a moving image in a mode in which the three-dimensional structure is graspable. Then, when the purchase reception unit 104 has received a purchase of prediction result data for the three-dimensional structure from the customer, the charging unit 105 executes charging processing with respect to the customer who has purchased the prediction result data. In this case, the customer determines whether the prediction result data for the three-dimensional structure is worth purchasing using the moving image output in the mode in which the three-dimensional structure of the protein is graspable as a determination material. Therefore, it is possible to charge the customer when the customer considers the prediction result data to be worth purchasing.

### [First Modification]

Next, a modification of the present example embodiment will be described in detail with reference to the drawings. Hereinafter, description overlapping with what has been described above will be omitted unless the omission obscures the description of the present example embodiment. The function of each component in each example embodiment of the present disclosure can be implemented not only by hardware similarly to the computer device illustrated in FIG. 2 but also by a computer device based on program control or software.

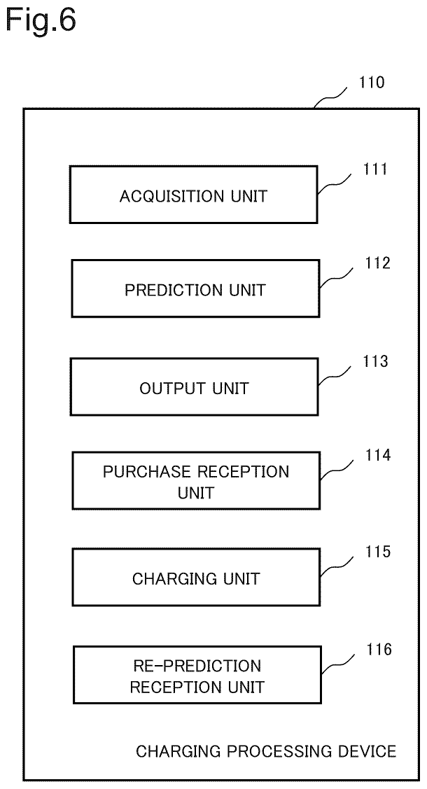

FIG. 6 is a diagram illustrating a configuration including a charging processing device 110 according to the present disclosure. With reference to FIG. 6, the charging processing device 110 will be described, focusing on the difference from the charging processing device 100.

The charging processing device 110 includes an acquisition unit 111, a prediction unit 112, an output unit 113, a purchase reception unit 114, a charging unit 115, and a re-prediction reception unit 116. The configurations of the acquisition unit 111, the prediction unit 112, the output unit 113, the purchase reception unit 114, and the charging unit 115 are basically the same as those of the corresponding components in the first example embodiment, and thus, description thereof is omitted.

In the present modification, when the customer is not satisfied with the prediction result for the three-dimensional structure, a re-prediction is received. Specifically, the re-prediction reception unit 116 receives a re-prediction of the three-dimensional structure. The re-prediction reception unit 116 may count the number of times a prediction for the same three-dimensional structure is received, and receive a re-prediction of the three-dimensional structure up to a predetermined number of times (for example, up to three times). When the input primary sequence data matches, under a predetermined condition, the primary sequence data used for predicting the three-dimensional structure in the past, the re-prediction reception unit 116 increases the number of times of reception by one time. For example, when a predetermined ratio (e.g., 80%) or more of a newly input primary sequence matches with a certain position of a primary sequence input in the past, or when a predetermined ratio (e.g., 80%) or more of a primary sequence input in the past matches with a certain position of a newly input primary sequence, the re-prediction reception unit 116 considers that a prediction for the same three-dimensional structure has been received. In this manner, in a case where the number of times of re-prediction is limited, it is possible to prevent a customer from unlimitedly using calculation resources for predicting a three-dimensional structure. When the customer purchases a prediction result of the three-dimensional structure after reaching the predetermined upper limit count number, the re-prediction reception unit 116 may reset the counted number.

When the re-prediction reception unit 116 receives a re-prediction of the three-dimensional structure, the prediction unit 112 re-predicts the three-dimensional structure with a changed prediction condition. Examples of the prediction condition include narrowing down the number of amino acid residues of the protein to be predicted. In the case where the number of amino acid residues of the protein to be predicted is narrowed down, for example, assuming that the number of amino acid residues of the protein of which the three-dimensional structure has been predicted first is 2000, the prediction unit 112 re-predicts the three-dimensional structure with the number of amino acid residues narrowed down to 1000 by ½. In this case, it is necessary to perform a prediction twice such as a prediction of a three-dimensional structure for the right half of the protein and a prediction of a three-dimensional structure for the left half of the protein. Therefore, the prediction unit 112 predicts the three-dimensional structure three times in total. As another method, a re-prediction may be performed with the number of amino acid residues narrowed down to 1000 by ½, and then a third prediction may be performed with the number of amino acid residues further narrowed down to 500 by ½ (the minimum required number of residues). In this case, the prediction unit 112 predicts the three-dimensional structure, for example, by narrowing down to a site of interest such as a highly unique site.

In a case where the protein to be predicted is a protein complex in which an antigen and an antibody are bound to each other, when the re-prediction reception unit 116 receives a re-prediction of the three-dimensional structure, the prediction unit 112 may predict the three-dimensional structure by narrowing down to a specific site having high commonality and a specific site having high uniqueness. In this case, the prediction unit 112 predicts the three-dimensional structure three times in total for the entire protein, for a highly common site, and for a highly unique site.

In the above-described example, the prediction condition for re-predicting the three-dimensional structure is changed by the prediction unit 112, but the re-prediction reception unit 116 may receive a condition for re-prediction. Specifically, the specification may be set in such a way that a site of the protein to be three-dimensionally predicted and the number of amino acid residues can be selected on a screen displaying a moving image of the three-dimensional structure of the protein.

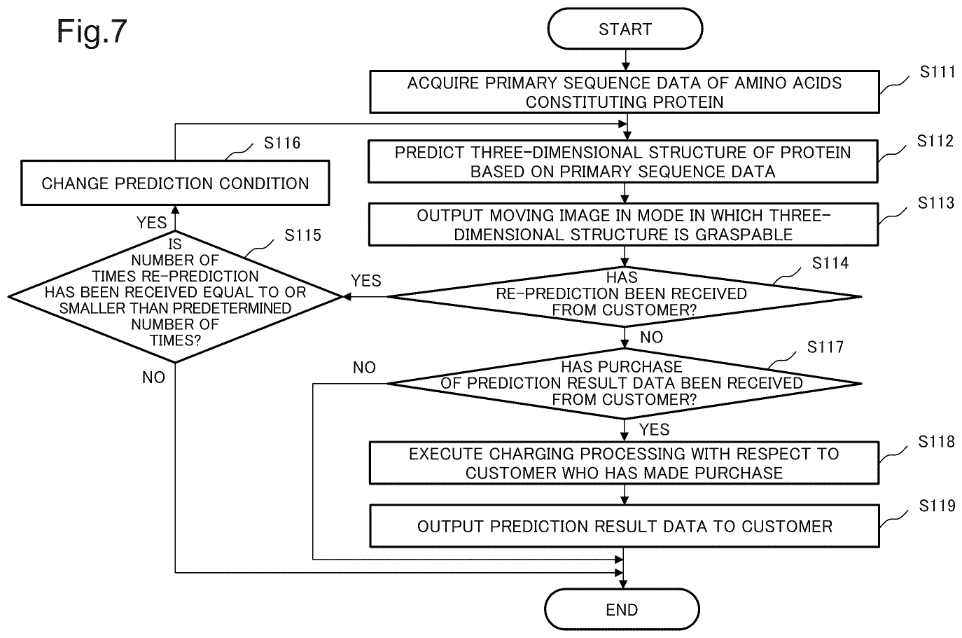

FIG. 7 is a flowchart illustrating an outline of an operation of the charging processing device 110 according to the modification. Note that the process according to this flowchart may be executed based on the program control by the processor described above.

As illustrated in FIG. 7, first, the acquisition unit 111 acquires primary sequence data of amino acids constituting a protein (step S111). Next, the prediction unit 112 predicts a three-dimensional structure of the protein based on the primary sequence data (step S112). Next, the output unit 113 outputs a moving image in a mode in which the three-dimensional structure is graspable (step S113). Next, when the re-prediction reception unit 116 has received a re-prediction of the three-dimensional structure from the customer (S114; YES), and when the number of times a re-prediction of the three-dimensional structure has been received is equal to or smaller than a predetermined number of times (S115; YES), the prediction unit 112 changes the prediction condition (step S116), and S112 and S113 are repeated. On the other hand, when the re-prediction reception unit 116 has received a re-prediction of the three-dimensional structure from the customer (S114; YES), and when the number of times a re-prediction of the three-dimensional structure has been received exceeds the predetermined number of times (S115; NO), the charging processing device 110 ends the flow.

Next, when the purchase reception unit 114 has received a purchase of a prediction result for the three-dimensional structure from the customer (S117; YES), the charging unit 115 execute charging processing with respect to the customer who has purchased the prediction result data (step S118), and the output unit 113 outputs the prediction result data to the customer (step S119). On the other hand, when the purchase reception unit 114 has not received a purchase of a prediction result for the three-dimensional structure from the customer (S117; NO), the charging processing device 110 ends the flow. In this manner, the charging processing device 110 ends the charging processing operation.

In the charging processing device 110, when the re-prediction reception unit 116 receives a re-prediction of the three-dimensional structure from the customer, the prediction unit 112 changes the prediction condition and repeats the prediction of the three-dimensional structure. Therefore, by performing a re-prediction with a changed condition, it is highly likely to show a three-dimensional structure considered to be worth by a customer.

### [Second Modification]

Next, another modification of the present example embodiment will be described in detail with reference to the drawings. Hereinafter, description overlapping with what has been described above will be omitted unless the omission obscures the description of the present example embodiment.

In the present modification, after outputting the prediction result data to the customer, the output unit 103 or the output unit 113 outputs information on structural reliability for the three-dimensional structure. The structural reliability is a degree of deviation of an estimated value from a true value of structural coordinates. The structural reliability may be obtained by scoring a degree of deviation of structural coordinates for each specific group between a predicted three-dimensional structure and a three-dimensional structure of teacher data.

The output unit 103 or the output unit 113 may output information on structure reliability for a location selected by the customer with a 3D viewer or the like capable of displaying the three-dimensional structure of the protein. In the present modification, the number of locations for which structural reliability is output may be limited in advance. In addition, the charging unit 105 or the charging unit 115 may perform additional charging processing with respect to the customer in a case where the information on structure reliability is output.

In the charging processing device according to the modification described above, the output unit 103 or the output unit 113 outputs information on structural reliability for the three-dimensional structure. As a result, the customer can grasp the reliability of the predicted three-dimensional structure.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

For example, although a plurality of operations is described in order in the form of a flowchart, the order in which the operations are described does not limit an order in which the plurality of operations is executed. Therefore, when each example embodiment is implemented, the order in which the plurality of operations is executed can be changed if the content is not affected by the change.

In a case where a three-dimensional structure of a protein is predicted using a protein structure prediction tool, there is a limit to a usage amount of a calculation server used for prediction, which limits the size of the protein that can be predicted. On the other hand, if charging is performed according to the usage amount of the calculation server used for prediction, even if the customer is not satisfied with the prediction result, an expensive cost is charged due to the use of the calculation server for a long period of time.

As an example of an effect of the present invention, it is possible to provide a charging processing device capable of charging a customer when the customer considers a prediction result to be worth purchasing.

Some or all of the above-described example embodiments may be described as in the following supplementary notes, but are not limited to the following supplementary notes.

### (Supplementary Note 1)

A charging processing device including:
an acquisition means for acquiring primary sequence data of amino acids constituting a protein;
a prediction means for predicting a three-dimensional structure of the protein based on the primary sequence data;
an output means for outputting a moving image in a mode in which the three-dimensional structure is graspable;
a purchase reception means for receiving a purchase of prediction result data for the three-dimensional structure from a customer; and
a charging means for executing charging processing with respect to the customer when the purchase is received.

### (Supplementary Note 2)

The charging processing device according to supplementary note 1, in which the moving image is a moving image obtained by rotating the three-dimensional structure around each of axes orthogonal to each other.

### (Supplementary Note 3)

The charging processing device according to supplementary note 1, in which the output means outputs the prediction result data to the customer when the purchase is received.

### (Supplementary Note 4)

The charging processing device according to supplementary note 1, further including
a re-prediction reception means for receiving a re-prediction of a three-dimensional structure after the moving image is output,
in which the prediction means predicts the three-dimensional structure of the protein with a changed condition when the re-prediction is received.

### (Supplementary Note 5)

The charging processing device according to supplementary note 4, in which the re-prediction reception means counts a number of times a prediction is received for a same three-dimensional structure and receives the re-prediction up to a predetermined number of times.

### (Supplementary Note 6)

The charging processing device according to supplementary note 5, in which the re-prediction reception means counts the number of times when the primary sequence data matches, under a predetermined condition, primary sequence data used for a prediction of a three-dimensional structure in the past.

### (Supplementary Note 7)

The charging processing device according to supplementary note 3, in which the output means further outputs structural reliability for up to a predetermined number of locations from the three-dimensional structure.

### (Supplementary Note 8)

The charging processing device according to supplementary note 1, in which the protein is a protein complex.

### (Supplementary Note 9)

A charging processing method performed by a computer, the charging processing method including:
acquiring primary sequence data of amino acids constituting a protein;
predicting a three-dimensional structure of the protein based on the primary sequence data;
outputting a moving image in a mode in which the three-dimensional structure is graspable;
receiving a purchase of prediction result data for the three-dimensional structure from a customer; and executing charging processing with respect to the customer when the purchase is received.

### (Supplementary Note 10)

A program for causing a computer to execute:
acquiring primary sequence data of amino acids constituting a protein;
predicting a three-dimensional structure of the protein based on the primary sequence data;
outputting a moving image in a mode in which the three-dimensional structure is graspable;
receiving a purchase of prediction result data for the three-dimensional structure from a customer; and
executing charging processing with respect to the customer when the purchase is received.

1. A charging processing device comprising:
a memory storing instructions; and
at least one processor configured to execute the instructions to:
acquire primary sequence data of amino acids constituting a protein;
predict a three-dimensional structure of the protein based on the primary sequence data;
output a moving image in a mode in which the three-dimensional structure is graspable;
receive a purchase of prediction result data for the three-dimensional structure from a customer; and
execute charging processing with respect to the customer when the purchase is received.

2. The charging processing device according to claim 1, wherein the moving image is a moving image obtained by rotating the three-dimensional structure around each of axes orthogonal to each other.

3. The charging processing device according to claim 1, wherein the at least one processor is further configured to execute the instructions to:
output the prediction result data to the customer when the purchase is received.

4. The charging processing device according to claim 1, wherein the at least one processor is further configured to execute the instructions to:
receive a re-prediction of a three-dimensional structure after the moving image is output; and
predict the three-dimensional structure of the protein with a changed condition when the re-prediction is received.

5. The charging processing device according to claim 4, wherein the at least one processor is further configured to execute the instructions to:
count a number of times a prediction is received for a same three-dimensional structure; and
receive the re-prediction up to a predetermined number of times.

6. The charging processing device according to claim 5, wherein the at least one processor is further configured to execute the instructions to:
count the number of times when the primary sequence data matches, under a predetermined condition, primary sequence data used for a prediction of a three-dimensional structure in the past.

7. The charging processing device according to claim 3, wherein the at least one processor is further configured to execute the instructions to:
output structural reliability for up to a predetermined number of locations from the three-dimensional structure.

8. The charging processing device according to claim 1, wherein the protein is a protein complex.

9. A charging processing method performed by a computer, the charging processing method comprising:
acquiring primary sequence data of amino acids constituting a protein;
predicting a three-dimensional structure of the protein based on the primary sequence data;
outputting a moving image in a mode in which the three-dimensional structure is graspable;
receiving a purchase of prediction result data for the three-dimensional structure from a customer; and
executing charging processing with respect to the customer when the purchase is received.

10. A non-transitory recording medium storing a program for causing a computer to execute:
acquiring primary sequence data of amino acids constituting a protein;
predicting a three-dimensional structure of the protein based on the primary sequence data;
outputting a moving image in a mode in which the three-dimensional structure is graspable;
receiving a purchase of prediction result data for the three-dimensional structure from a customer; and
executing charging processing with respect to the customer when the purchase is received.
